(19) United States
(12) Patent Application Publication
(10) Pub. No.: US 2013/0331575 A1
(43) Pub. Date: Dec. 12, 2013

(54) Method for resolution of 4-((1R,3S)-6-chloro-3-phenyl-indan-1-yl)-1,2,2-trimethyl-piperazine and 1-((1R,3S)-6-chloro-3-phenyl-indan-1-yl)-3,3-dimethyl-piperazine

(71) Applicant: Robert Dancer, Hvidovre (DK)

(72) Inventor: Robert Dancer, Hvidovre (DK)

(21) Appl. No.: 13/933,198

(22) PCT Filed: Jan. 6, 2012
(86) PCT No.: PCT/EP2012/050174

§ 371 Date: Aug. 28, 2013

Related U.S. Application Data
(60) Provisional application No. 61/430,552, filed on Jan. 7, 2011.

(30) Foreign Application Priority Data
Jan. 7, 2011 (DK) PA 2011 00011

(51) Int. Cl. C07D 241/04

(52) U.S. Cl. CPC C07D 241/04
USPC 544/403

(57) ABSTRACT

The present invention relates to resolution methods for manufacture of 4-((1R,3S)-6-chloro-3-phenyl-indan-1-yl)-1,2,2-trimethyl-piperazine and 1-((1R,3S)-6-chloro-3-phenyl-indan-1-yl)-3,3-dimethyl-piperazine and pharmaceutically acceptable salts thereof.

### FIELD OF THE INVENTION

The present invention relates to resolution methods for manufacture of 4-((1R,3S)-6-chloro-3-phenyl-indan-1-yl)-1,2,2-trimethyl-piperazine and 1-((1R,3S)-6-chloro-3-phenyl-indan-1-yl)-3,3-dimethyl-piperazine and pharmaceutically acceptable salts thereof.

### BACKGROUND

The compounds of the present invention 4-((1R,3S)-6-chloro-3-phenyl-indan-1-yl)-1,2,2-trimethyl-piperazine (I) and 1-((1R,3S)-6-chloro-3-phenyl-indan-1-yl)-3,3-dimethyl-piperazine (II) hereinafter referred to as Compound (I) and (II) have the respective molecular structures depicted below.

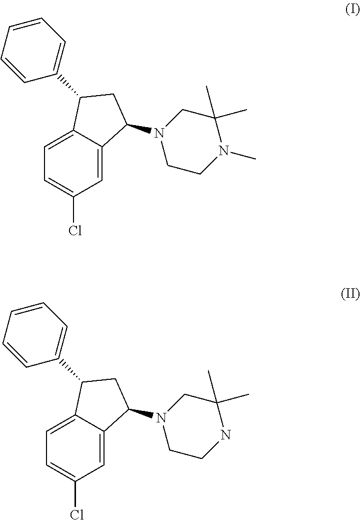 [SMILES: CN1CCN([C@@H]2C[C@@H](c3ccccc3)c3ccc(Cl)cc32)CC1(C)C ; CC1(C)CN([C@@H]2C[C@@H](c3ccccc3)c3ccc(Cl)cc32)CCN1]

A group of trans isomers of 3-aryl-1-(1-piperazinyl)indanes substituted in the 2- and/or 3-position of the piperazine ring has been described in WO 93/22293 and in Klaus P. Bøgesø, Drug Hunting, the Medicinal Chemistry of 1-piperazino-3-phenylindans and Related Compounds, 1998, ISBN 87-88085-10-4 (cf, e.g. compound 69 in table 3, p. 47 and in table 9A, p. 101). The compounds are described as having high affinity for dopamine D₁ and D₂ receptors and the 5-HT₂ receptor and are suggested to be useful for treatment of several diseases in the central nervous system, including schizophrenia.

Trans racemic 4-((6-chloro-3-phenyl-indan-1-yl)-1,2,2-trimethyl-piperazine and trans racemic 1-(6-chloro-3-phenyl-indan-1-yl)-3,3-dimethyl-piperazine may e.g. be synthesized analogously to the methods outlined in Bøgesø et al., J. Med. Chem., 1995, 38, p. 4380-4392 and in WO 93/22293. Manufacture of Compound (I) by resolution of trans racemic 4-((6-chloro-3-phenyl-indan-1-yl)-1,2,2-trimethyl-piperazine has been described by Bøgesø et al. in J. Med. Chem., 1995, 38, p. 4380-4392, see table 5, compound (−)-38. The process described comprises the use of (+)-ditoluoyl tartaric acid for resolution in ethylacetate, and Compound (I) is isolated as the fumarate salt.

The synthesis of Compound (II) from optically pure starting materials has been described in WO 2005/016900, WO 2005/016901 and WO 2006/086984. Synthesis of Compound (I) from Compound (II) by N-alkylation is disclosed in WO 2005/016900 (p. 31, example 12). A crystalline hydrogen tartrate salt of Compound (II) has been disclosed in WO 2006/086985.

Bøgesø et al., J. Med. Chem., 1995, 38, p. 4380-4392 discloses that Compound (I) is a potent D₁/D₂ antagonists showing some D₁ selectivity in vitro while in vivo it is equipotent as D₁ and D₂ antagonist. The compound is also described as a potent 5-HT₂ antagonist and as having high affinity for α₁ adrenoceptors. As disclosed in WO 2005/016901 Compound (II) displays a similar receptor profile and pharmacological activity as Compound (I).

### SUMMARY OF THE INVENTION

The present inventors have found that a high yield and a high enantiomeric excess of 4-((1R,3S)-6-chloro-3-phenyl-indan-1-yl)-1,2,2-trimethyl-piperazine and 1-((1R,3S)-6-chloro-3-phenyl-indan-1-yl)-3,3-dimethyl-piperazine can be obtained by resolution of their respective racemates by the careful selection of a suitable enantiomerically pure acid and a solvent.

In particular, for the resolution of 4-((1R,3S)-6-chloro-3-phenyl-indan-1-yl)-1,2,2-trimethyl-piperazine from the corresponding racemate the present inventors have found that combination of dibenzoyl-L-tartaric acid or (S)-Chlorophos with a solvent selected from the group consisting of 2-butanone (MEK), ethyl acetate (EtOAc) and acetonitrile (ACN) give surprisingly high enantiomeric excesses (ee) and good crystallinities.

Correspondingly, for the resolution of 1-((1R,3S)-6-chloro-3-phenyl-indan-1-yl)-3,3-dimethyl-piperazine from the corresponding racemate the present inventors have found that combination of diisopropylidene-2-keto-L-gulonic acid or (S)-(+)-1,1′-binaphtyl-2,2′-diyl hydrogenphosphate with a solvent selected from the group consisting of methanol (MeOH), ethyl acetate (EtOAc) and acetonitrile (ACN) give surprisingly high enantiomeric excesses (ee) and good crystallinities.

The resolution methods of the present invention have been found to provide a yield of at least about 30% under certain circumstances up to more than 45% which is strikingly higher than the yield obtained by the resolution method described in Bøgesø et al., J. Med. Chem., 1995, 38, p. 4380-4392 wherein (+)-ditoluoyl tartaric acid is used for resolution of trans racemic 4-(6-chloro-3-phenyl-indan-1-yl)-1,2,2-trimethyl-piperazine.

The problem set out to be solved by the present invention is the resolution of trans racemic 4-(6-chloro-3-phenyl-indan-1-yl)-1,2,2-trimethyl-piperazine and trans racemic 1-(6-chloro-3-phenyl-indan-1-yl)-3,3-dimethyl-piperazine) into the respective enantiomeric compounds, 4-((1R,3S)-6-chloro-3-phenyl-indan-1-yl)-1,2,2-trimethyl-piperazine and 1-((1R,3S)-6-chloro-3-phenyl-indan-1-yl)-3,3-dimethyl-piperazine. In a preferred embodiment the enantiomeric excess is at least about 30%, either in the solid phase (resolution) or in the liquid phase (reverse resolution). In a preferred embodiment the enantiomeric compounds, i.e. 4-((1R,3S)-6-chloro-3-phenyl-indan-1-yl)-1,2,2-trimethyl-piperazine and 1-((1R,3S)-6-chloro-3-phenyl-indan-1-yl)-3,3-dimethyl-piperazine, respectively, are crystallized in the solid phase.

The resolution of trans-racemic 4-(6-chloro-3-phenyl-indan-1-yl)-1,2,2-trimethyl-piperazine was also attempted with other selections of acid and solvent than those covered by the present invention. However, these alternatives suffer from a low enantiomeric excess in the product.

Likewise, trans racemic 1-(6-chloro-3-phenyl-indan-1-yl)-3,3-dimethyl-piperazin) was attempted resolved with other selections of acid and solvent with the same disadvantages.

Accordingly, in brief, the present invention relates to processes wherein the racemate is mixed with an enantiomerically pure acid in a solvent. The mixture may optionally be heated to an appropriate temperature to obtain a solution of the racemate and the enantiomerically pure acid. Subsequent precipitation of the enantiomers may be obtained e.g. by cooling or evaporation and the precipitate may be isolated and optionally dried. It is the experience of the inventors that recrystallisation of the precipitate may increase the enantiomeric excess. The choice of solvent and conditions for the resolution process e.g. temperature and stoichiometry of the starting materials may be used to optimize the yield and enantiomeric excess of the desired enantiomer.

The present invention clearly also covers the process of reverse resolution where the antipode of trans-4-((1R,3S)-6-chloro-3-phenylindan-1-yl)-1,2,2-trimethyl piperazine or the antipode of trans-1-((1R,3S)-6-chloro-3-phenyl-indan-1-yl)-3,3-dimethyl-piperazine is crystallised in high ee. In case of reverse resolution 4-((1R,3S)-6-chloro-3-phenylindan-1-yl)-1,2,2-trimethyl piperazine or trans-1-((1R,3S)-6-chloro-3-phenyl-indan-1-yl)-3,3-dimethyl-piperazine can be isolated from the liquid phase, e.g. in the form of a salt or a free base.

### DEFINITIONS

The term “trans-4-((1R,3S)-6-chloro-3-phenylindan-1-yl)-1,2,2-trimethyl piperazine” or “4-((1R,3S)-6-chloro-3-phenylindan-1-yl)-1,2,2-trimethyl piperazine” corresponds to the enantiomer Compound (I).

The term “trans-4-((1S,3R)-6-chloro-3-phenylindan-1-yl)-1,2,2-trimethyl piperazine” or “4-((1S,3R)-6-chloro-3-phenylindan-1-yl)-1,2,2-trimethyl piperazine” corresponds to the antipode of Compound (I).

The term “trans-1-((1R,3S)-6-chloro-3-phenyl-indan-1-yl)-3,3-dimethyl-piperazine” or “1-((1R,3S)-6-chloro-3-phenyl-indan-1-yl)-3,3-dimethyl-piperazine” corresponds to the enantiomer Compound (II).

The term “trans-1-((1S,3R)-6-chloro-3-phenyl-indan-1-yl)-3,3-dimethyl-piperazine” or “1-((1S,3R)-6-chloro-3-phenyl-indan-1-yl)-3,3-dimethyl-piperazine” corresponds to the antipode of Compound (II).

As used herein, the term “trans racemic 4-((6-chloro-3-phenyl-indan-1-yl)-1,2,2-trimethyl-piperazine” refers to the racemate of 4-((1R,3S)-6-chloro-3-phenylindan-1-yl)-1,2,2-trimethyl piperazine and 4-((1S,3R)-6-chloro-3-phenylindan-1-yl)-1,2,2-trimethyl piperazine. The same principle applies for “trans racemic 1-(6-chloro-3-phenyl-indan-1-yl)-3,3-dimethyl-piperazine”.

In the present context, a “racemate” refers to an equal mixture of non-superimposable mirror images.

In the present context, the term “trans-4-(6-chloro-3-phenylindan-1-yl)-1,2,2-trimethyl-piperazine”, i.e. without any specific indication of the enantiomer form (e.g. using (+) and (−), or using the R/S-convention) refers to a mixture of the two enantiomers, 4-((1R,3S)-6-chloro-3-phenylindan-1-yl)-1,2,2-trimethyl piperazine and 4-((1S,3R)-6-chloro-3-phenylindan-1-yl)-1,2,2-trimethyl piperazine. The same principle applies for the “trans-1-(6-chloro-3-phenyl-indan-1-yl)-3,3-dimethyl-piperazine”.

In the context of the present invention resolution shall also cover the process of reverse resolution.

In the present context, “yield” is calculated base on the total mass of the salts of the racemate in the process; it is thereby understood that the maximum yield of a pure enantiomer can not exceed 50% when starting from a racemate.

As described herein, Compound (I) and Compound (II) respectively, is intended to designate any form of the compound, such as the free base, pharmaceutically acceptable salts thereof, e.g. pharmaceutically acceptable acid addition salts, such as succinate and malonate salts, hydrates or solvates of the free base or salts thereof, as well as anhydrous forms, amorphous forms, or crystalline forms.

As described herein, the term “enantiomerically pure acid” is defined as an acid in which at least 95% of the enantiomeric part of the acid is one of a pair of non-superimposable mirror images.

As described herein “a pharmaceutically acceptable salt” of a compound of Formula I or II includes pharmaceutically acceptable acid addition salts. Acid addition salts include salts of inorganic acids as well as organic acids. Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like.

In the context of the present invention the terms “resolution” and “reverse resolution” describes a process by which a racemate is separated into its two enantiomers.

In the present context, heating to an “appropriate temperature” indicates that the composition is heated to a temperature suitable for obtaining a solution, such as above room temperature such as above 40° C., such as above 45° C., such as above 50° C., such as above 55° C., such as above 60° C., such as above 65° C., such as above 70° C. limited by the reflux temperature of the solvent. Dependent on the solvent used “appropriate temperature” might indicate reflux temperature, i.e. the composition is heated at reflux.

In the present context, “reflux” is a technique involving the condensation of vapors and the return of this condensate to the system from which it originated.

In the present context, “recrystallization” is a procedure for purifying compounds. Recrystallization can be performed by e.g. single-solvent recrystallization, multi-solvent recrystallization or hot filtration-recrystallization.

In the present context, “enantiomeric excess” is abbreviated ee and defined as the absolute difference between the mole fractions of each enantiomer of a compound.

### DETAILED DESCRIPTION OF THE INVENTION

The present invention relates to a process for the manufacture of 4-((1R,3S)-6-chloro-3-phenyl-indan-1-yl)-1,2,2-trimethyl-piperazine (Compound (I)) comprising resolution of trans-4-((6-chloro-3-phenyl-indan-1-yl)-1,2,2-trimethyl-piperazine with suitable enantiomerically pure acid in the presence of a solvent.

Accordingly, the present invention relates in a first embodiment (E1) to a process for the manufacture of 4-((1R,3S)-6-chloro-3-phenyl-indan-1-yl)-1,2,2-trimethyl-piperazine (Compound (I)) comprising resolution of trans-4-((6-chloro-3-phenyl-indan-1-yl)-1,2,2-trimethyl-piperazine with suitable enantiomerically pure acid in the presence of a solvent, wherein the enantiomerically pure acid is selected from the group consisting of dibenzoyl-L-tartaric acid, (S)-chlorophos, dibenzoyl-D-tartaric acid and (R)-chlorophos.

In a second embodiment (E2) of (E1) the solvent comprises at least 30% of one or more of the solvents selected from the group consisting of C₃-C₈ ketones, C₁-C₅ esters of acetic acid, C₁-C₅ esters of propiotic acid, C₁-C₄ alcohols and C₂-C₃ nitriles. In preferred embodiments of (E2) the solvent comprises at least 35% or more, such as at least 40%, 50%, 60%, 70%, 80%, 90%, or 95% or 100% of one or more of the solvents selected from the group consisting of C₃-C₈ ketones, C₁-C₅ esters of acetic acid, C₁-C₅ esters of propionic acid, C₁-C₄ alcohols and C₂-C₃ nitriles.

In a third embodiment (E3) the solvent of the process of any of embodiment (E1) or (E2) is selected from the group consisting of 2-butanone (MEK), ethyl acetate (EtOAc) and acetonitrile (ACN).

In a further embodiment (E4) of any of embodiment (E1), (E2), or E(3) the enantiomerically pure acid is Dibenzoyl-L-tartaric and the solvent is acetonitrile; or the enantiomerically pure acid is Dibenzoyl-L-tartaric and the solvent is 2-butanone; or the enantiomerically pure acid is Dibenzoyl-L-tartaric and the solvent is ethyl acetate, or the enantiomerically pure acid is Dibenzoyl-D-tartaric and the solvent is acetonitrile; or the enantiomerically pure acid is Dibenzoyl-D-tartaric and the solvent is 2-butanone; or the enantiomerically pure acid is Dibenzoyl-D-tartaric and the solvent is ethyl acetate.

In a further embodiment (E5) any of embodiment (E1), (E2), or E(3) the enantiomerically pure acid is (S)-Chlorophos and the solvent is acetonitrile; or the enantiomerically pure acid is (S)-Chlorophos and the solvent is 2-butanone; or the enantiomerically pure acid is (S)-Chlorophos and the solvent is ethyl acetate, or the enantiomerically pure acid is (R)-Chlorophos and the solvent is acetonitrile; or the enantiomerically pure acid is (R)-Chlorophos and the solvent is 2-butanone; or the enantiomerically pure acid is (R)-Chlorophos and the solvent is ethyl acetate.

In a preferred embodiment (E6) of any of embodiment (E1), (E2), or E(3) the solvent is acetonitrile and the enantiomerically pure acid is (S)-Chlorophos.

In another preferred embodiment (E7) of any of embodiment (E1), (E2), or E(3) the solvent is acetonitrile and the enantiomerically pure acid is dibenzoyl-L-tartaric acid.

In an embodiment (E8) the process of embodiment (E1) comprises the steps of
a) mixing trans-4-(6-chloro-3-phenyl-indan-1-yl)-1,2,2-trimethyl-piperazine and the enantiomerically pure acid in a solvent;
b) optionally heating the obtained mixture to an appropriate temperature to obtain a solution of the trans-4-(6-chloro-3-phenyl-indan-1-yl)-1,2,2-trimethyl-piperazine and the enantiomerically pure acid;
c) optionally cooling the solution obtained in b) until precipitation;
d) isolating the precipitate obtained in step a), b), or c);
e) optionally drying the precipitate obtained in d);
f) optionally isolating 4-((1R,3S)-6-chloro-3-phenyl-indan-1-yl)-1,2,2-trimethyl-piperazine at an appropriate temperature from the liquid obtained after step d) if the precipitate obtained in step a), b), or c) is a salt of 4-((1S,3R)-6-chloro-3-phenyl-indan-1-yl)-1,2,2-trimethyl-piperazine;
   to obtain 4-((1R,3S)-6-chloro-3-phenyl-indan-1-yl)-1,2,2-trimethyl-piperazine or a salt thereof, preferably a pharmaceutically acceptable salt. Optionally, the process comprises a subsequent step in which the precipitate is recrystallised after step d) or e) or f).

In an embodiment (E9) of the process of any of the previous embodiment E(1)-E(8) the isolated 4-((1R,3S)-6-chloro-3-phenyl-indan-1-yl)-1,2,2-trimethyl-piperazine is an intermediate.

In an embodiment (E10) of the process of any of the previous embodiment E(1)-E(8) the formed salt of 4-((1R,3S)-6-chloro-3-phenyl-indan-1-yl)-1,2,2-trimethyl-piperazine is the end product of the process.

In an embodiment (E11) of any of the previous embodiments the salt of 4-((1R,3S)-6-chloro-3-phenyl-indan-1-yl)-1,2,2-trimethyl-piperazine is a pharmaceutically acceptable salt, wherein the pharmaceutically acceptable salt preferably is selected from the list of pharmaceutically acceptable salt in the section Definitions of the present application.

In an embodiment (E12) of the embodiment (E9) the isolated 4-((1R,3S)-6-chloro-3-phenyl-indan-1-yl)-1,2,2-trimethyl-piperazine is in the form of a salt which is dissolved in a solvent and recrystallized as a pharmaceutically acceptable salt, wherein the pharmaceutically acceptable salt preferably is selected from the list of pharmaceutically acceptable salt in the section Definitions of the present application.

In an embodiment (E13) of the process of embodiment (E8) the appropriate temperature of step b) is about 40° C. or higher, such as about 45° C., preferably about 50° C. or about 55° C. or higher such as about 60° C., such as about 65° C., such as about 70° C.

In an embodiment (E14) of the process of embodiment (E8) step c), the solution is cooled to a temperature of about 25° C. or lower, such as about 20° C., or 15° C., preferably about 10° C. or lower, such as about 5° C. or 0° C.

The present invention also relates to a process for manufacture of 1-((1R,3S)-6-chloro-3-phenyl-indan-1-yl)-3,3-dimethyl-piperazine (Compound (II)) comprising resolution of trans-1-(6-chloro-3-phenyl-indan-1-yl)-3,3-dimethyl-piperazine with suitable enantiomerically pure acid in the presence of a solvent.

Accordingly, the present invention relates in an embodiment (E15) to a process for the manufacture of 1-((1R,3S)-6-chloro-3-phenyl-indan-1-yl)-3,3-dimethyl-piperazine comprising resolution of trans-1-(6-chloro-3-phenyl-indan-1-yl)-3,3-dimethyl-piperazine with suitable enantiomerically pure acid in the presence of a solvent, wherein the enantiomerically pure acid is selected from the group consisting of diisopropylidene-2-keto-L-gulonic acid, diisopropylidene-2-keto-D-gulonic acid, (S)-(+)-1,1′-binaphthyl-2,2′-diyl hydrogenphosphate, (R)-(−)-1,1′-binaphthyl-2,2′-diyl hydrogenphosphate, (R)-chlorophos, (S)-chlorophos, dibenzoyl-L-tartaric acid, dibenzoyl-D-tartaric acid and camphoric acid.

In a further embodiment (E16) the solvent comprises at least 30% of one or more of the solvents selected from the group consisting of C₃-C₈ ketones, C₁-C₅ esters of acetic acid, C₁-C₅ esters of propiotic acid, C₁-C₄ alcohols and C₂-C₃ nitriles. In preferred embodiments of (E16) the solvent comprises at least 35% or more, such as at least 40%, 50%, 60%, 70%, 80%, 90%, or 95% or 100% of one or more of the solvents selected from the group consisting of C₃-C₈ ketones, C₁-C₅ esters of acetic acid, C₁-C₅ esters of propiotic acid, C₁-C₄ alcohols and C₂-C₃ nitriles

In a further embodiment (E17) the solvent of the process of any of embodiment (E15) and (E16) is selected from the group consisting of 2-butanone (MEK), ethyl acetate (EtOAc), methanol (McOH) and acetonitrile (ACN).

In a further embodiment (E18) of the process of (E15) the enantiomerically pure acid is dibenzoyl-L-tartaric and the solvent is acetonitrile; or the enantiomerically pure acid is diisopropylidene-2-keto-L-gulonic acid and the solvent is methanol; or the enantiomerically pure acid is diisopropylidene-2-keto-L-gulonic acid and the solvent is acetonitrile; or the enantiomerically pure acid is (S)-(+)-1,1′-binaphthyl-2,2′-diyl hydrogenphosphate and the solvent is ethyl acetate; or the enantiomerically pure acid is (S)-Chlorophos and the solvent is ethyl acetate; or the enantiomerically pure acid is N-acetyl-L-leucine and the solvent is ethyl acetate; or the enantiomerically pure acid is N-acetyl-L-leucine and the solvent is acetonitrile; or the enantiomerically pure acid is D-Quinic acid and the solvent is ethyl acetate; or the enantiomerically pure acid is (R)-Chlorophos and the solvent is 2-butanone; or the enantiomerically pure acid is camphoric acid and the solvent is acetonitrile, or the enantiomerically pure acid is diisopropylidene-2-keto-D-gulonic acid and the solvent is methanol; or the enantiomerically pure acid is diisopropylidene-2-keto-D-gulonic acid and the solvent is acetonitrile; or the enantiomerically pure acid is (R)-(−)-1,1′-binaphthyl-2,2′-diyl hydrogenphosphate and the solvent is ethyl acetate; or the enantiomerically pure acid is (R)-Chlorophos and the solvent is ethyl acetate; or the enantiomerically pure acid is N-acetyl-D-leucine and the solvent is ethyl acetate; or the enantiomerically pure acid is N-acetyl-D-leucine and the solvent is acetonitrile; or the enantiomerically pure acid is L-Quinic acid and the solvent is ethyl acetate; or the enantiomerically pure acid is (S)-Chlorophos and the solvent is 2-butanone; or the enantiomerically pure acid is camphoric acid and the solvent is acetonitrile.

In a preferred embodiment (E19) of (E15) or (E16) the enantiomerically pure acid is diisopropylidene-2-keto-L-gulonic acid and the solvent is methanol.

In a preferred embodiment (E20) of (E15) or (E16) the enantiomerically pure acid is diisopropylidene-2-keto-L-gulonic acid and the solvent is acetonitrile.

In another preferred embodiment (E21) of (E15) or (E16) the enantiomerically pure acid is (S)-(+)-1,1′-binaphthyl-2,2′-diyl hydrogenphosphate and the solvent is ethyl acetate.

In an embodiment (E22) the process of embodiment (E15) or (E16) comprising the steps of
a) mixing trans-1-(6-chloro-3-phenyl-indan-1-yl)-3,3-dimethyl-piperazine and the enantiomerically pure acid in a solvent;
b) optionally heating the obtained mixture to an appropriate temperature to obtain a solution of the trans-1-(6-chloro-3-phenyl-indan-1-yl)-3,3-dimethyl-piperazine and the enantiomerically pure acid;
c) optionally cooling the solution obtained in b) until precipitation;
d) isolating the precipitate obtained in step a), b), or c);
e) optionally drying the precipitate obtained in d);
f) optionally isolating 1-((1R,3S)-6-chloro-3-phenyl-indan-1-yl)-3,3-dimethyl-piperazine at an appropriate temperature from the liquid obtained after step d) if the precipitate obtained in step a), b), or c) is a salt of 1-((1S,3R)-6-chloro-3-phenyl-indan-1-yl)-3,3-dimethyl-piperazine;
   to obtain 1-((1R,3S)-6-chloro-3-phenyl-indan-1-yl)-3,3-dimethyl-piperazine or a salt thereof, preferably a pharmaceutically acceptable salt. Optionally, the process comprises a subsequent step in which the precipitate is recrystallised after step d) or e) or f).

In a further embodiment (E23) the isolated 1-((1R,3S)-6-chloro-3-phenyl-indan-1-yl)-3,3-dimethyl-piperazine obtained in any of embodiments (E15) to (E22), optionally in the form of a salt, is methylated to obtain 4-((1R,3S)-6-chloro-3-phenyl-indan-1-yl)-1,2,2-trimethyl-piperazine.

In an embodiment (E24) of the process of any of the previous embodiment E(15)-E(22) the isolated 1-((1R,3S)-6-chloro-3-phenyl-indan-1-yl)-3,3-dimethyl-piperazine is an intermediate.

In an embodiment (E25) of the process of any of the previous embodiment E(15)-E(22) the formed salt of 1-((1R,3S)-6-chloro-3-phenyl-indan-1-yl)-3,3-dimethyl-piperazine is the end product of the process.

In an embodiment (E26) of the process of any of the previous embodiment E(15)-E(22) the salt of 1-((1R,3S)-6-chloro-3-phenyl-indan-1-yl)-3,3-dimethyl-piperazine is a pharmaceutically acceptable salt, wherein the pharmaceutically acceptable salt preferably is selected from the list of pharmaceutically acceptable salt in the section Definitions of the present application.

In an embodiment (E27) of the embodiment (E24) the isolated 1-((1R,3S)-6-chloro-3-phenyl-indan-1-yl)-3,3-dimethyl-piperazine is in the form of a salt which is dissolved in a solvent and recrystallized as a pharmaceutically acceptable salt, wherein the pharmaceutically acceptable salt preferably is selected from the list of pharmaceutically acceptable salt in the section Definitions of the present application.

In an embodiment (E28) of the process of embodiment (E22) the appropriate temperature of step b) is about 40° C. or higher, such as about 45° C., preferably about 50° C. or about 55° C. or higher such as about 60° C., such as about 65° C., such as about 70° C.

In an embodiment (E29) of the process of embodiment (E22) step c), the solution is cooled to a temperature of about 25° C. or lower, such as about 20° C., or 15° C., preferably about 10° C. or lower, such as about 5° C. or 0° C.

All references cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate). The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The invention will be illustrated in the following non-limiting examples.

### EXPERIMENTAL

### Instrument and Methodology Details

### X-Ray Powder Diffraction (XRPD)

X-Ray Powder Diffraction patterns were collected on a Bruker D8 diffractometer using Cu Kα radiation (40 kV, 40 mA), θ-2θ goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.5.0 and the data were analysed and presented using Diffrac Plus EVA v11.0.0.2 or v13.0.0.2.

Samples were run under ambient conditions as flat plate specimens using powder as received. The sample was gently packed into a cavity cut into polished, zero-background (510) silicon wafer. The sample was rotated in its own plane during analysis. The details of the data collection are:
Angular range: 2 to 42 °2θ
Step size: 0.05 °2θ
Collection time: 0.5 s/step

### Nuclear Magnetic Resonance (NMR)

### ¹H NMR

NMR spectra were collected on a Bruker 400 MHz instrument equipped with an auto-sampler and controlled by a DRX400 console. Automated experiments were acquired using ICON-NMR v4.0.4 running with Topspin v1.3 using the standard Bruker loaded experiments. For non-routine spectroscopy, data were acquired through the use of Topspin alone.

Samples were prepared in DMSO-d6, unless otherwise stated. Off-line analysis was carried out using ACD SpecManager v12.00.

### Differential Scanning Calorimetry (DSC)

DSC data were collected on a TA Instruments Q2000 equipped with a 50 position auto-sampler. The calibration for thermal capacity was carried out using sapphire and the calibration for energy and temperature was carried out using certified indium.

Typically 0.5-1.5 mg of each sample, in a pin-holed aluminium pan, was heated at 10° C./min from 25° C. to 250-350° C. A purge of dry nitrogen at 50 ml/min was maintained over the sample.

The instrument control software was Advantage for Q Series v2.8.0.392 and Thermal Advantage v4.8.3 and the data were analysed using Universal Analysis v4.4A.

The instrument control and data analysis software was STARe v9.20.

### Thermo-Gravimetric Analysis (TA)

TGA data were collected on a TA Instruments Q500 TGA, equipped with a 16 position auto-sampler. The instrument was temperature calibrated using certified Alumel and Nickel. Typically 5-10 mg of each sample was loaded onto a pre-tared aluminium DSC pan and heated at 10° C./min from ambient temperature to 250-350° C. A nitrogen purge at 60 ml/min was maintained over the sample.

The instrument control software was Advantage for Q Series v2.8.0.392 and Thermal Advantage v4.8.3 and the data were analysed using Universal Analysis v4.4A.

### Chemical Purity Determination by HPLC

Purity analysis was performed on an Agilent HP1100 series system equipped with a diode array detector and using ChemStation software vB.02.01-SR1 using the method detailed below:

TABLE 1
HPLC Method Parameters for Chemical Purity Determinations

| Sample Preparation | 0.5-1 mg/mL in acetonitrile:water 1:1 | | |
|---|---|---|---|
| Column | Supelco Ascentis Express C18, | | |
| | 100 × 4.6 mm, 2.7 μm | | |
| Column Temperature (° C.) | 25 | | |
| Injection (microL) | 10 | | |
| Detection: | 255, 90 nm | | |
| Wavelength, Bandwidth (nm) | | | |
| Flow Rate (mL/min) | 2.0 | | |
| Phase A | 0.1% TFA in water | | |
| Phase B | 0.085% TFA in acetonitrile | | |
| | Time (min) | % Phase A | % Phase B |
| Timetable | 0 | 95 | 5 |
| | 6 | 5 | 95 |
| | 6.2 | 95 | 5 |
| | 8 | 95 | 5 |

### Chiral Purity Determination by HPLC

Chiral purity was performed on a Hewlett Packard 1100 series system equipped with a diode array detector and using ChemStation for LC Rev. A.08.03[847].

TABLE 2
HPLC method parameters for chiral purity determination
(30 minute method)

| Sample Preparation | 1-3 mg/mL in Hexane/IPA (90/10 v/v) |
|---|---|
| Column: | Chiralpak ADH 5microm 250 × 4.6 mm |
| Column Temperature (° C.): | 30 |
| Injection (microL): | 5 |
| Detection: | 240, 8 |
| Wavelength, Bandwidth (nm): | |
| Flow Rate (mL · min⁻¹): | 0.6 |
| Mobile Phase | Hexane/IPA/DEA/Propionic acid |
| | 90/10/0.2/2 |

### HPLC Methods:

The chiral purity of trans-4-((1R,3S)-6-chloro-3-phenyl-indan-1-yl)-1,2,2-trimethyl-piperazine was measured by chiral HPLC chromatography as described above.

The retention times for the two enantiomers were 8.5-8.6 min for the (1S,3R) enantiomer and 13.6-13.7 min for Compound (I).

The chiral purity of trans-1-((1R,3S)-6-chloro-3-phenyl-indan-1-yl)-3,3-dimethyl-piperazine was measured by chiral HPLC as described above.

The retention times for the two enantiomers were 9.9-10.1 min for the (1S,3R) enantiomer and 16.1-16.4 min for Compound (II).

Each salt was free-based prior to be analysed by chiral HPLC. The filtered solid salt (2-3 mg) was dissolved in DCM (0.4 mL) at RT and aqueous NaOH 1 M solution (0.2 mL) was added. The resulting DCM layer was withdrawn and fully evaporated under reduced pressure (to dryness).

The dry residue obtained (free-base) was dissolved in hexane/IPA (90:10 v/v) prior to be analysed by chiral HPLC.

### Example 1

### Resolution of 4-((1R,3S)-6-chloro-3-phenyl-indan-1-yl)-1,2,2-trimethyl-piperazine using (R,R)-dibenzoyl-L-tartaric acid (L-BDT)

General procedure: To a mixture of trans racemic 4-((1R,3S)-6-chloro-3-phenyl-indan-1-yl)-1,2,2-trimethyl-piperazine (0.5 g, 1.4 mmol) and (R,R)-dibenzoyl-L-tartaric acid (0.5 g, 1.4 mmol) was added a solvent (5 mL), and the mixture was warmed with stirring until a homogeneous solution was obtained. The solutions were allowed to cool to room temperature, and were stirred for 24 hours. The solids obtained were removed by filtration, dried, and the yields and chiral purities were determined (see table 1). The samples were purified by reslurrying in solvent (5 mL) at room temperature for 24 hours. The samples were then filtered and dried, and the yields and chiral purities were determined.

TABLE 1
Chiral purity of Compound I

| | | | First crystallisation | | Reslurry | |
|---|---|---|---|---|---|---|
| Racemic | | Solvent | | Chiral | | Chiral |
| Zicron- | | (5 mL) | | purity | | purity |
| apine | L-DBT | (mixtures | Yield | (e.r.) | Yield | (e.r.) |
| (g) | (g) | are v:v) | (g) | (%) | (g) | (%) |
| 0.5 | 0.5 | ACN | 0.56 | 75.4 | 0.27 | 84.1 |
| 0.5 | 0.5 | ACN/H2O | 0.3 | 78.5 | 0.2 | 93.3 |
| | | 9:1 | | | | |
| 0.5 | 0.5 | ACN/H2O | 0.45 | 80.1 | 0.31 | 90.1 |
| | | 8:2 | | | | |
| 0.5 | 0.5 | iso-Propyl | 0.76 | 49.6 | 0.41 | 51.7 |
| | | acetate | | | | |
| 0.5 | 0.5 | Ethylformate | 0.68 | 56.3 | 0.51 | 52.3 |
| 0.5 | 0.5 | Acetone | 0.46 | 88.2 | 0.28 | 89.7 |
| 0.5 | 0.5 | DMF/H2O | No solid observed | | | |
| | | 1:1 | | | | |
| 0.5 | 0.5 | Propionitrile | 0.47 | 77.5 | 0.3 | 85.5 |
| 0.5 | 0.5 | Iso-propyl | 0.61 | 66.5 | 0.41 | 78.9 |
| | | alcohol | | | | |
| 0.5 | 0.5 | EtOH/H2O | 0.88 | 53.9 | 0.64 | 51.2 |
| | | 1:1 | | | | |

### Example 2

### 4-((1R,3S)-6-chloro-3-phenyl-indan-1-yl)-1,2,2-trimethyl-piperazine (S)-Chlorophos salt, acetonitrile

Trans racemic 4-(6-chloro-3-phenyl-indan-1-yl)-1,2,2-trimethyl-piperazine (200 mg) was suspended in acetonitrile (4 mL) and heated to 50° C. under stirring.

A suspension of(S)-Chlorophos, prepared in acetonitrile (2 mL) at room temperature (20-25° C.), was slowly added (dropwise) to the warm suspension of free-base. The system was left under stirring, at 50° C., until a clear solution was obtained.

After 5-10 minutes, the solution was seeded with less than 1 mg of crystalline trans-4-(6-chloro-3-phenyl-indan-1-yl)-1,2,2-trimethyl-piperazine mono(S)-Chlorophos salt (ee=93% of trans-4-((1R,3S)-6-chloro-3-phenyl-indan-1-yl)-1,2,2-trimethyl-piperazine).

A precipitation started within a few minutes and additional acetonitrile (2 mL) was added to the suspension obtained.

The system was then subjected to a cooling ramp from 50° C. to 0° C. at 0.1° C./min, then held at 0° C. for 3 hours prior to be heated to 25° C. at 2° C./minute.

The suspension was left under stirring at 25° C. for 65 hours, and the product was filtered under vacuum. The resulting fresh filtered cake was washed with acetonitrile (2 mL) prior to be dried in the hood at room temperature. Yield 155.9 mg (43.8%). The content of the two enantiomers were (1S,3R) enantiomer=0.6% and (1R,3S) enantiomer=99.4% corresponding to an ee=98.8% of trans-4-((1R,3S)-6-chloro-3-phenyl-indan-1-yl)-1,2,2-trimethyl-piperazine (S)-Chlorophos salt.

The dry product (145 mg) was suspended in acetonitrile (1 mL) at room temperature (20-25° C.) under stirring for 2 days and the solid was filtered under vacuum. The resulting fresh filtered cake was washed with acetonitrile (0.5 mL) prior to be dried in the hood at room temperature. Yield 139 mg (39%).

The content of the two enantiomers were (1S,3R)enantiomer=0.3% and (1R,3S)enantiomer=99.7% corresponding to an ee=99.4% of trans-4-((1R,3S)-6-chloro-3-phenyl-indan-1-yl)-1,2,2-trimethyl-piperazine mono(S)-Chlorophos salt. High resolution XRPD confirmed the crystalline nature of the product.

1H NMR spectrum was consistent with the stoichiometry 1:1 of a mono-(S)-Chlorophos salt, confirmed by comparison of integrals from counter-ion and trans-4-(6-chloro-3-phenyl-indan-1-yl)-1,2,2-trimethyl-piperazine signals.

1H-NMR (DMSO) δ: 0.65 (3H, s), 0.92 (3H, s), 1.20-1.40 (7H, br m), 1.99 (1H br s), 2.30-2.42 (1H, br m), 2.53-2.82 (5H, m), 2.94 (1H, br s), 3.13 (2H, br s), 3.49 (1H, dd), 4.06 (1H, d), 4.45 (2H, dt), 5.54 (1H, d), 6.97 (1H, d), 7.10 (2H, d), 7.21 (1H, tt), 7.26-7.42 (7H, m), 7.46 (1H, dd), 10.80 (1H, br d).

Melting point=196-198° C. Chemical purity=98.6% area.

### Example 3

### 4-((1R,3S)-6-chloro-3-phenyl-indan-1-yl)-1,2,2-trimethyl-piperazine dibenzoyl-L-tartrate, acetonitrile

Trans racemic 4-(6-chloro-3-phenyl-indan-1-yl)-1,2,2-trimethyl-piperazine (200 mg) was suspended in acetonitrile (4 mL) and heated to 50° C. under stirring.

A solution of dibenzoyl-L-tartaric acid, prepared in acetonitrile (3 mL) at room temperature (20-25° C.), was slowly added (dropwise) to the warm suspension of free-base. The system was left under stirring, at 50° C., until a clear solution was obtained. After 5-10 minutes, the solution was seeded with less than 1 mg of crystalline trans-4-(6-chloro-3-phenyl-indan-1-yl)-1,2,2-trimethyl-piperazine mono dibenzoyl-L-tartrate (ee=72.6% of trans-4-((1R,3S)-6-chloro-3-phenyl-indan-1-yl)-1,2,2-trimethyl-piperazine).

The system was then subjected to a cooling ramp from 50° C. to 0° C. at 0.1° C./min, then held at 0° C. for 3 hours prior to be heated to 25° C. at 2° C./minute.

The suspension was left under stirring at 25° C. for 65 hours, and the product was filtered under vacuum. The resulting fresh filtered cake was washed with acetonitrile (3 mL) prior to be dried in the hood at room temperature. Yield 189 mg (47.0%).

The content of the two enantiomers were (1S,3R) enantiomer-6.3% and (1R,3S) enantiomer=93.7% corresponding to an ee=87.4% of trans-4-((1R,3S)-6-chloro-3-phenyl-indan-1-yl)-1,2,2-trimethyl-piperazine dibenzoyl-L-tartrate.

The dry product (175 mg) was suspended in acetonitrile (1 mL) at 25° C. under stirring for 2 days and the solid was filtered under vacuum. The resulting fresh filtered cake was washed with acetonitrile (1 mL) prior to be dried in the hood at room temperature. Yield 152 mg (37.8%).

The content of the two enantiomers were (1S,3R)enantiomer=4.6% and (1R,3S)enantiomer=95.4% corresponding to an ee=90.8% of trans-4-((1R,3S)-6-chloro-3-phenyl-indan-1-yl)-1,2,2-trimethyl-piperazine mono dibenzoyl-L-tartrate 8). High resolution XRPD) confirmed the crystalline nature of the product. 1H NMR spectrum was consistent with the stoichiometry 1:1 of a mono-dibenzoyl-L-tartrate salt, confirmed by comparison of integrals from counter-ion and trans-4-(6-chloro-3-phenyl-indan-1-yl)-1,2,2-trimethyl-piperazine signals.

Melting point=155-158° C. Chemical purity=98.1% area.

### Example 4

### 1-(1S,3R)-6-chloro-3-phenyl-indan-1-yl)-3,3-dimethyl-piperazine Diisopropylidene-2-keto-L-gulonate, methanol

Trans racemic 1-(6-chloro-3-phenyl-indan-1-yl)-3,3-dimethyl-piperazine (200 mg) was dissolved in methanol (4 mL) at 25° C. under stirring.

A solution of diisopropylidene-2-keto-L-gulonic acid monohydrate (171.5 mg), prepared in methanol (2 mL) at room temperature (20-25° C.), was slowly added (dropwise) to the clear solution of free-base.

After 5-10 minutes, the solution was seeded with less than 1 mg of crystalline trans-1-(6-chloro-3-phenyl-indan-1-yl)-3,3-dimethyl-piperazine mono diisopropylidene-2-keto-L-gulonate (ee=92.0% of trans-1-((1S,3R)-6-chloro-3-phenyl-indan-1-yl)-3,3-dimethyl-piperazine). The system was then subjected to a cooling ramp from 25° C. to 0° C. at

0.1° C./min, then held at 0° C. for 3 hours prior to be heated to 25° C. at 2° C./minute. The suspension obtained was left under stirring at 25° C. for 65 hours, and the product was filtered under vacuum. The resulting fresh filtered cake was washed with methanol
(1 mL) prior to be dried in the hood at room temperature. Yield 72.8 mg (19.6%). The content of the two enantiomers were (1R,3S) enantiomer=1.9% and (1S,3R) enantiomer=98.1% corresponding to an ee=96.2% of trans-1-((1S,3R)-6-chloro-3-phenyl-indan-1-yl)-3,3-dimethyl-piperazine diisopropylidene-2-keto-L-gulonate.

The dry product (70 mg) was suspended in methanol (0.55 mL) at 25° C. under stirring for 2 days and the solid was filtered under vacuum. The resulting fresh filtered cake was washed with methanol (0.4 mL) prior to be dried in the hood at room temperature. Yield 54 mg (14.5%).

The content of the two enantiomers were (1R,3S) enantiomer=0.6% and (1S,3R) enantiomer=99.4% corresponding to an ee=98.8% of trans-1-((1S,3R)-6-chloro-3-phenyl-indan-1-yl)-3,3-dimethyl-piperazine mono diisopropylidene-2-keto-L-gulonate.

High resolution XRPD confirmed the crystalline nature of the product. 1H NMR spectrum was consistent with the stoichiometry 1:1 of a mono diisopropylidene-2-keto-L-gulonate salt, confirmed by comparison of integrals from counter-ion and trams-1-(6-chloro-3-phenyl-indan-1-yl)-3,3-dimethyl-piperazine signals.

Melting point=201-203° C. Chemical purity=97.7% area.

### Example 5

### 1-((1R,3S)-6-chloro-3-phenyl-indan-1-yl)-3,3-dimethyl-piperazine Diisopropylidene-2-keto-L-gulonate, methanol

Trans racemic 1-(6-chloro-3-phenyl-indan-1-yl)-3,3-dimethyl-piperazine (400 mg) was dissolved in methanol (3 mL) at 25° C. under stirring.

A solution of diisopropylidene-2-keto-L-gulonic acid monohydrate (343.0 mg), prepared in methanol (3 mL) at room temperature (20-25° C.), was slowly added (dropwise) to the clear solution of free-base.

After 5-10 minutes, the solution was seeded with less than 1 mg of crystalline trans-1-(6-chloro-3-phenyl-indan-1-yl)-3,3-dimethyl-piperazine mono diisopropylidene-2-keto-L-gulonate (ee=92.0% of trans-1-((1S,3R)-6-chloro-3-phenyl-indan-1-yl)-3,3-dimethyl-piperazine). The system was then subjected to a cooling ramp from 25° C. to 0° C. at

0.1° C./min, then held at 0° C. for 3 hours prior to be heated to 25° C. at 2° C./minute.

The suspension obtained was left under stirring at 25° C. for 65 hours, and the product was filtered under vacuum. The resulting filtered solid was dried in the hood at room temperature. Yield 290 mg (35%).

The content of the two enantiomers were (1R,3S) enantiomer=13.5% and (1S,3R) enantiomer 86.5% corresponding to an ee=73% of trans-1-((1S,3R)-6-chloro-3-phenyl-indan-1-yl)-3,3-dimethyl-piperazine diisopropylidenc-2-keto-L-gulonate.

The resulting filtered solution (4.51 g) was evaporated at room temperature in the hood to dryness. Yield 410 mg (55.2%) of dry residue.

The content of the two enantiomers were (1S,3R) enantiomer=26.2% and (1R,3S) enantiomer=73.8% corresponding to an ee=47.6% of trans-1-((1R,3S)-6-chloro-3-phenyl-indan-1-yl)-3,3-dimethyl-piperazine diisopropylidene-2-keto-L-gulonate.

The later dry residue (350 mg) was suspended in methyl-tert-butyl-ether (5 mL) at room temperature (20-25° C.) under stirring for 24 hours and the solid was filtered under vacuum. The resulting fresh filtered cake was washed with methyl-tert-butyl-ether (0.5 mL) prior to be dried in the hood at room temperature. Yield 309 mg (41.6%).

The content of the two enantiomers were (1S,3R) enantiomer=22.1% and (1R,3S) enantiomer=77.9% corresponding to an ee=55.8% of trans-1-((1R,3S)-6-chloro-3-phenyl-indan-1-yl)-3,3-dimethyl-piperazine mono diisopropylidene-2-keto-L-gulonate.

High resolution XRPD confirmed the crystalline nature of the product. 1H NMR spectrum was consistent with the stoichiometry 1:1 of a mono diisopropylidene-2-keto-L-gulonate salt, confirmed by comparison of integrals from counter-ion and trans-1-(6-chloro-3-phenyl-indan-1-yl)-3,3-dimethyl-piperazine signals.

Decomposition starting above 137° C. Chemical purity=98.6% area.

### Example 6

### 1-((1R,3S)-6-chloro-3-phenyl-indan-1-yl)-3,3-dimethyl-piperazine Diisopropylidenc-2-keto-L-gulonate, acetonitrile

Trans racemic 1-(6-chloro-3-phenyl-indan-1-yl)-3,3-dimethyl-piperazine (200 mg) was dissolved in acetonitrile (4 mL) at 25° C. under stirring.

A suspension of diisopropylidene-2-keto-L-gulonic acid monohydrate (171.5 mg), prepared in acetonitrile (3 mL) at room temperature (20-25° C.), was slowly added (dropwise) and the suspension was heated to 50-60° C. until all diisopropylidene-2-keto-L-gulonic acid had dissolved.

The warm solution was left for cooling at room temperature prior to be seeded with less than 1 mg of crystalline trans-1-(6-chloro-3-phenyl-indan-1-yl)-3,3-dimethyl-piperazine mono diisopropylidene-2-keto-L-gulonate (ee=33.1% of trans-1-((1R,3S)-6-chloro-3-phenyl-indan-1-yl)-3,3-dimethyl-piperazine).

The system was then left under stirring at room temperature (20-25° C.) for 24 hours and the solid precipitate was filtered under vacuum. The resulting fresh filtered cake was washed with acetonitrile (0.5 mL) prior to be dried in the hood at room temperature. Yield 97.3 mg (26.2%).

The content of the two enantiomers were (1S,3R) enantiomer=5.1% and (1S,3R) enantiomer=94.9% corresponding to an ee=89.8% of trans-1-((1R,3S)-6-chloro-3-phenyl-indan-1-yl)-3,3-dimethyl-piperazine diisopropylidene-2-keto-L-gulonate.

The dry product (90 mg) was suspended in acetonitrile (0.6 mL) at room temperature under stirring for 24 hours and the solid was filtered under vacuum. The resulting fresh filtered cake was washed with acetonitrile (0.2 mL) prior to be dried in the hood at room temperature. Yield 80.5 mg (21.7%).

The content of the two enantiomers were (1S,3R) enantiomer=1.3% and (1S,3R) enantiomer=98.7% corresponding to an ee=97.4% of trans-1-((1R,3S)-6-chloro-3-phenyl-indan-1-yl)-3,3-dimethyl-piperazine mono diisopropylidene-2-keto-L-gulonate.

High resolution XRPD confirmed the crystalline nature of the product. 1H NMR spectrum was consistent with the stoichiometry 1:1 of a mono diisopropylidenc-2-keto-L-gulonate salt, confirmed by comparison of integrals from counter-ion and trans-1-(6-chloro-3-phenyl-indan-1-yl)-3,3-dimethyl-piperazine signals.

Decomposition starting above 143° C. Chemical purity=98.6% area.

### Example 7

### 1-((1R,3S)-6-chloro-3-phenyl-indan-1-yl)-3,3-dimethyl-piperazine (S)-(+)-1,1′-binaphthyl-2,2′-diyl hydrogenphosphate salt, ethyl acetate

Trans racemic 1-(6-chloro-3-phenyl-indan-1-yl)-3,3-dimethyl-piperazine (200 mg) was dissolved in ethyl acetate (6 mL) at 50° C. under stirring.

A suspension of(S)-(+)-1,1′-binaphthyl-2,2′-diyl hydrogenphosphate (204.3 mg), prepared in ethyl acetate (3 mL) at room temperature (20-25° C.), was slowly added (dropwise) to the warm solution of free-base. The system was left under stirring at 50° C. until the acid had completely dissolved.

After 5-10 minutes, the clear solution obtained was seeded with less than 1 mg of crystalline trans-1-(6-chloro-3-phenyl-indan-1-yl)-3,3-dimethyl-piperazine mono(S)-(+)-1,1′-binaphthyl-2,2′-diyl hydrogenphosphate salt (ee=84.2% of trans-1-((1R,3S)-6-chloro-3-phenyl-indan-1-yl)-3,3-dimethyl-piperazine).

A precipitation started within a few minutes and additional ethyl acetate (2 mL) was added to the suspension obtained.

The system was then subjected to a cooling ramp from 50° C. to 0° C. at 0.1° C./min, then held at 0° C. for 3 hours prior to be heated to 25° C. at 2° C./minute.

The suspension was left under stirring at 25° C. for 65 hours, and the product was filtered under vacuum. The resulting fresh filtered cake was washed with ethyl acetate (2 mL) prior to be dried in the hood at room temperature. Yield 190.5 mg (47.1%). The content of the two enantiomers were (1S,3R) enantiomer=3.5% and (1R,3S) enantiomer=96.5% corresponding to an ee=93% of trans-1-((1R,3S)-6-chloro-3-phenyl-indan-1-yl)-3,3-dimethyl-piperazine (S)-(+)-1,1′-binaphthyl-2,2′-diyl hydrogenphosphate salt.

The dry product (165 mg) was suspended in ethyl acetate (1.5 mL) at 25° C. under stirring for 2 days and the solid was filtered under vacuum. The resulting fresh filtered cake was washed with ethyl acetate (1 mL) prior to be dried in the hood at room temperature. Yield 143 mg (35.4%).

The content of the two enantiomers were (1S,3R) enantiomer=1.9% and (1R,3S) enantiomer=98.1% corresponding to an ee=96.2% of trans-1-((1R,3S)-6-chloro-3-phenyl-indan-1-yl)-3,3-dimethyl-piperazine (S)-(+)-1,1′-binaphthyl-2,2′-diyl hydrogenphosphate salt.

High resolution XRPD confirmed the crystalline nature of the product. 1H NMR spectrum was consistent with the stoichiometry 1:1 of a mono(S)-(+)-1,1′-binaphthyl-2,2′-diyl hydrogenphosphate salt, confirmed by comparison of integrals from counter-ion and trans-1-(6-chloro-3-phenyl-indan-1-yl)-3,3-dimethyl-piperazine signals.

Melt/decomposition=318° C. Chemical purity=99.9% area.

1. A process for the manufacture of 4-((1R,3S)-6-chloro-3-phenyl-indan-1-yl)-1,2,2-trimethyl-piperazine or a salt thereof comprising resolution of trans-4-((6-chloro-3-phenyl-indan-1-yl)-1,2,2-trimethyl-piperazine with an enantiomerically pure acid in the presence of a solvent, wherein the enantiomerically pure acid is selected from the group consisting of dibenzoyl-L-tartaric acid, (S)-Chlorophos, dibenzoyl-D-tartaric acid and (R)-Chlorophos.

2. The process according to claim 1 wherein the solvent comprises at least 30% of one or more of the solvents selected from the group consisting of C₃-C₈ ketones, C₁-C₅ esters of acetic acid, C₁-C₅ esters of propiotic acid, C₁-C₄ alcohols and C₂-C₃ nitriles.

3. The process according to claim 2 wherein the solvent is selected from the group consisting of 2-butanone, ethyl acetate and acetonitrile.

4. The process according to claim 1 wherein the enantiomerically pure acid is (S)-Chlorophos.

5. The process according to claim 1 further comprising the steps of
a) mixing trans-4-(6-chloro-3-phenyl-indan-1-yl)-1,2,2-trimethyl-piperazine and the enantiomerically pure acid in a solvent;
b) optionally heating the obtained mixture to an appropriate temperature to obtain a solution of the trans-4-(6-chloro-3-phenyl-indan-1-yl)-1,2,2-trimethyl-piperazine and the enantiomerically pure acid;
c) optionally cooling the solution obtained in b) until precipitation;
d) isolating the precipitate obtained in step a), b), or c);
e) optionally drying the precipitate obtained in d);
f) optionally isolating 4-((1R,3S)-6-chloro-3-phenyl-indan-1-yl)-1,2,2-trimethyl-piperazine at an appropriate temperature from the liquid obtained after step d) if the precipitate obtained in step a), b), or c) is a salt of 4-((1S,3R)-6-chloro-3-phenyl-indan-1-yl)-1,2,2-trimethyl-piperazine;
to obtain 4-((1R,3S)-6-chloro-3-phenyl-indan-1-yl)-1,2,2-trimethyl-piperazine or a salt thereof.

6. A process for the manufacture of 1-((1R,3S)-6-chloro-3-phenyl-indan-1-yl)-3,3-dimethyl-piperazine comprising resolution of trans-1-(6-chloro-3-phenyl-indan-1-yl)-3,3-dimethyl-piperazine with an enantiomerically pure acid in the presence of a solvent, wherein the enantiomerically pure acid is selected from the group consisting of diisopropylidene-2-keto-L-gulonic acid, diisopropylidene-2-keto-D-gulonic acid, (S)-(+)-1,1′-binaphthyl-2,2′-diyl hydrogenphosphate, (R)-(−)-1,1′-binaphthyl-2,2′-diyl hydrogenphosphate, (R)-Chlorophos, (S)-Chlorophos, dibenzoyl-L-tartaric acid, dibenzoyl-D-tartaric acid and camphoric acid.

7. The process according to claim 6 wherein the solvent comprises at least 30% of one or more of the solvents selected from the group consisting of C₃-C₈ ketones, C₁-C₅ esters of acetic acid, C₁-C₅ esters of propiotic acid, C₁-C₄ alcohols and C₂-C₃ nitriles.

8. The process according to claim 7 wherein the solvent is selected from the group consisting of 2-butanone, ethyl acetate, methanol and acetonitrile.

9. The process according to claim 8 wherein the solvent is acetonitrile.

10. The process according to claim 6 further comprising the steps of
a) mixing trans-1-(6-chloro-3-phenyl-indan-1-yl)-3,3-dimethyl-piperazine and the enantiomerically pure acid in a solvent;
b) optionally heating the obtained mixture to an appropriate temperature to obtain a solution of the trans 1-(6-chloro-3-phenyl-indan-1-yl)-3,3-dimethyl-piperazine and the enantiomerically pure acid;
c) optionally cooling the solution obtained in b) until precipitation;
d) isolating the precipitate obtained in step a), b), or c);
e) optionally drying the precipitate obtained in d);
f) optionally isolating 1-((1R,3S)-6-chloro-3-phenyl-indan-1-yl)-3,3-dimethyl-piperazine at an appropriate temperature from the liquid obtained after step d) if the precipitate obtained in step a), b), or c) is a salt of 1-((1S,3R)-6-chloro-3-phenyl-indan-1-yl)-3,3-dimethyl-piperazine;
to obtain 1-((1R,3S)-6-chloro-3-phenyl-indan-1-yl)-3,3-dimethyl-piperazine or a salt thereof.

11. A process for the manufacture of trans-4-((1R,3S)-6-chloro-3-phenyl-indan-1-yl)-1,2,2-trimethyl-piperazine or a salt thereof comprising methylation of trans-1-((1R,3S)-6-chloro-3-phenyl-indan-1-yl)-3,3-dimethyl-piperazine obtained by the process of claim 6.

12. The process according to claim 4 wherein the solvent is selected from the group consisting of acetonitrile, 2-butanone and ethyl acetate.

13. The process according to claim 1 wherein the enantiomerically pure acid is dibenzoyl-L-tartaric acid.

14. The process according to claim 13 wherein the solvent is selected from the group consisting of acetonitrile, 2-butanone and ethyl acetate.

15. The process according to claim 5 further comprising recrystallizing the precipitate after step d), step e) or step f).

16. The process according to claim 9 wherein the enantiomerically pure acid is selected from the group consisting of dibenzoyl-L-tartaric, diisopropylidene-2-keto-L-gulonic acid and camphoric acid.

17. The process according to claim 8 wherein the enantiomerically pure acid is diisopropylidene-2-keto-L-gulonic acid and the solvent is methanol.

18. The process according to claim 8 wherein the enantiomerically pure acid is (S)-(+)-1,1′-binaphthyl-2,2′-diyl hydrogenphosphate and the solvent is ethyl acetate.

19. The process according to claim 8 wherein the enantiomerically pure acid is (R)-Chlorophos and the solvent is 2-butanone.

20. The process according to claim 10 further comprising recrystallizing the precipitate after step d), step e) or step f).
